(19) United States
(12) Patent Application Publication
(10) Pub. No.: US 2022/0296754 A1
(43) Pub. Date: Sep. 22, 2022

(54) Folding UV Array

(71) Applicant: X Development LLC, Mountain View, CA (US)

(72) Inventors: Marc Strauss, Fremont, CA (US); Joshua Seal, San Jose, CA (US)

(21) Appl. No.: 17/204,193

(22) Filed: Mar. 17, 2021

(51) Int. Cl. A61L 2/24; B25J 15/00; A61L 2/10

(52) U.S. Cl. CPC A61L 2/24; B25J 15/0019; A61L 2/10; A61L 2202/11; A61L 2202/14

(57) ABSTRACT

An end effector of a robotic device is disclosed. The end effector includes a plurality of array segments, a plurality of UV light modules, and a first articulating member. Each array segment of the plurality of array segments is coupled to at least one other array segment of the plurality of array segments. Moreover, each UV light module is coupled to a different array segment of the plurality of array segments. The first articulating member is configured to cause at least one of the plurality of array segments to move relative to at least one other array segment.

### BACKGROUND

As technology advances, various types of robotic devices are being created for performing a variety of functions that may assist users. Robotic devices may be used for applications involving material handling, transportation, welding, assembly, and dispensing, among others. Over time, the manner in which these robotic systems operate is becoming more intelligent, efficient, and intuitive. As robotic systems become increasingly prevalent in numerous aspects of modern life, it is desirable for robotic systems to be efficient. Therefore, a demand for efficient robotic systems has helped open up a field of innovation in actuators, movement, sensing techniques, as well as component design and assembly.

### SUMMARY

Example embodiments involve a mobile robot that uses an ultraviolet (UV) illuminator to emit UV light towards different features of an environment in order to sanitize certain portions of the environment. Specifically, the mobile robot may include an array of UV modules that emit UV light towards surfaces of an environment. The array is foldable such that non-planar surfaces, such as door handles, can be more effectively sanitized.

In an embodiment, an end effector of a robotic device is disclosed. The end effector includes a plurality of array segments. Each array segment of the plurality of array segments is coupled to at least one other array segment. Additionally, the end effector includes a plurality of UV light modules. The UV light modules may be configured to use UV radiation to sanitize surfaces, among other examples. Each UV light module is coupled to a different array segment of the plurality of array segments. As such, in some embodiments, the end effector may be considered to have an array of UV light modules. The end effector also includes a first articulating member that is configured to cause at least one of the plurality of array segments to move relative to at least one other array segment. In some examples, the first articulating member may rotate relative to a housing or body of the end effector and cause the plurality of UV light modules to go from a first alignment to a second alignment. Further, in some additional examples, the array of UV light modules may be considered a foldable array of UV light modules. As such, in some regards, the motion of one UV light module relative another may be considered a folding motion.

In another embodiment, a method is disclosed. The method includes a first articulating member of an end effector of a robotic device causing a plurality of UV light modules to be in a first alignment relative to one another. Among other possibilities, the first alignment may be a reference alignment or a starting alignment. Each UV light module is coupled to one of a plurality of array segments. Moreover, the plurality of array segments is coupled to the first articulating member. The method also includes the first articulating member moving at least one of the plurality of array segments such that at least a portion of the plurality of UV light modules are rotated into a second alignment relative to one another. In some examples, the method may also include a sensor determining contours of a surface and then adjusting the positioning of at least one of the plurality of UV light modules accordingly such that the UV light modules are in a sanitizing alignment corresponding to the particular surface. In even other examples, the method may include sanitizing a surface using the plurality of UV light modules of the end effector.

In a further embodiment a robotic system is disclosed. The robotic system includes a sensor and an end effector. The end effector is configured to sanitize surfaces. Moreover, the end effector includes a plurality of array segments, a plurality of UV light modules, and a first articulating member. Each array segment is coupled to at least one other array segment and each UV light module is coupled to one array segment. Moreover, in some examples, the array segments are able to move relative one another in what may be described as a folding motion. As such, the end effector may be considered to include a foldable array of UV light modules. The first articulating member is configured to cause at least one of the plurality of array segments to move relative to at least one other array segment. The robotic system further includes circuitry configured to perform a variety of operations. The operations which the circuitry is configured to perform includes determining contours of a surface to be sanitized based on sensor data from the sensor and operating the first articulating member to move at least one of the plurality of array segments. The movement of plurality of array segments is specific such that the plurality of UV light modules are aligned relative to one another based on the determined contours of the surface to be sanitized.

In further aspects, any type of robotic system or device could be used or configured as a means for performing any of the methods described herein (or portions of the methods described herein). For example, a robotic system including an end effector includes means to operate the end effector.

The foregoing summary is illustrative only and is not intended to be in any way limiting. In addition to the illustrative aspects, embodiments, and features described above, further aspects, embodiments, and features will become apparent by reference to the figures and the following detailed description and the accompanying drawings.

### BRIEF DESCRIPTION OF THE DRAWINGS

FIG. 1 illustrates a configuration of a robotic system, in accordance with example embodiments.

FIG. 2 illustrates a mobile robot, in accordance with example embodiments.

FIG. 3 illustrates an exploded view of a mobile robot, in accordance with example embodiments.

FIG. 4 illustrates a robotic arm, in accordance with example embodiments.

FIG. 5A illustrates an end effector with a foldable UV-light array, in accordance with example embodiments.

FIG. 5B illustrates an end effector with a foldable UV-light array, in accordance with example embodiments.

FIG. 5C illustrates an end effector with a foldable UV-light array, in accordance with example embodiments.

FIG. 5D illustrates an end effector with a foldable UV-light array, in accordance with example embodiments.

FIG. 6A illustrates an end effector with a foldable UV-light array, in accordance with example embodiments.

FIG. 6B illustrates an end effector with a foldable UV-light array, in accordance with example embodiments.

FIG. 7A illustrates an end effector with a foldable UV-light array coupled to a mobile robot, in accordance with example embodiments.

FIG. 7B illustrates an end effector with a foldable UV-light array coupled to a mobile robot, in accordance with example embodiments.

FIG. 8 illustrates a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless indicated as such. Other embodiments can be utilized, and other changes can be made, without departing from the scope of the subject matter presented herein.

Thus, the example embodiments described herein are not meant to be limiting. It will be readily understood that the aspects of the present disclosure, as generally described herein, and illustrated in the figures, can be arranged, substituted, combined, separated, and designed in a wide variety of different configurations.

Throughout this description, the articles “a” or “an” are used to introduce elements of the example embodiments. Any reference to “a” or “an” refers to “at least one,” and any reference to “the” refers to “the at least one,” unless otherwise specified, or unless the context clearly dictates otherwise. The intent of using the conjunction “or” within a described list of at least two terms is to indicate any of the listed terms or any combination of the listed terms.

The use of ordinal numbers such as “first,” “second,” “third” and so on is to distinguish respective elements rather than to denote a particular order of those elements. For the purpose of this description, the terms “multiple” and “a plurality of” refer to “two or more” or “more than one.”

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment. In the figures, similar symbols typically identify similar components, unless context dictates otherwise. Further, unless otherwise noted, figures are not drawn to scale and are used for illustrative purposes only. Moreover, the figures are representational only and not all components are shown. For example, additional structural or restraining components might not be shown.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. OVERVIEW

Described herein is an example robotic device along with example operations that may be performed by the example robotic device and/or variations thereof. The example robotic device may include a number of components coupled together, including a mobile base, an arm, an end of arm system (EOAS), a midsection, a mast, and a perception housing. The arm may include particular degrees of freedom (DOFs), ranges of motion (ROMs), joint types, link lengths, and joint offsets to optimize the performance of tasks. Tradeoffs exist in terms of desired operational capabilities of the robot relative to space constraints and cost constraints. Some example robots described herein are engineered to simplify manufacturing and programming, making the robots affordable for non-industrial applications.

In general, a robotic device described herein may be used in a plurality of settings and may be configured to perform operations corresponding to each setting. For example, the robotic device may be used as a household aid robot. The robotic device may be configured to clean and sanitize various surfaces, collect and load laundry into a hamper or washer, clean the floor by collecting garbage, clean up toys by gathering the toys and loading them into a toy storage bin, move pieces of furniture into their proper positions, and fetch drinks, food, and keys, among other possible tasks. The robotic device may additionally be used as a yard work aid robot to perform certain yard work such as sweeping up and gathering fallen leaves, sticks, and any other undesirable items that may be left in a back or front yard. The robotic device may be configured to perform any of the operations described herein autonomously in order to reduce an amount of human input needed to control the robotic device.

An example robot device may include an end effector specifically configured for sanitizing surfaces. Particularly, one example implementation of end effector may incorporate UV lamps configured to emit UV radiation to sanitize a surface exposed to the UV radiation. While using UV light for sanitization may be known, effectively sanitizing surfaces, specifically including (but not limited to non-planar surfaces) remains a challenge.

In order to effectively sanitize a surface using UV radiation, the intensity of UV light, the proximity (or distance between) of the UV lamp to the surface, and time of exposure to the UV should be considered. Moreover, it may be more effective to directly expose a surface to the UV radiation, but that may be difficult for non-planar surfaces. For example, using a linear wand-type UV light over a planar surface may provide some sanitizing effect, but in order to sanitize a non-planar surface, such as a door handle or railing a linear wand-type UV light might need to make multiple passes to cover all the sides and/or features of the surface.

Furthermore, exposure to intense UV radiation may be detrimental to humans and as such it may be preferred to utilize robotic technologies in using UV light to sanitize surfaces. However, in order to be effective, a robot configured to clean or sanitize a surface must have the controls and features available to effectively and in a cost effective manner expose the surface to UV radiation. As such, for example, a robot system for sanitizing surfaces may include an end effector that is configured to surround a surface with or otherwise adjust the relative positioning of multiple UV light modules so that all or nearly all features of a surface are exposed to sanitizing UV radiation.

### II. EXAMPLE ROBOTIC SYSTEMS

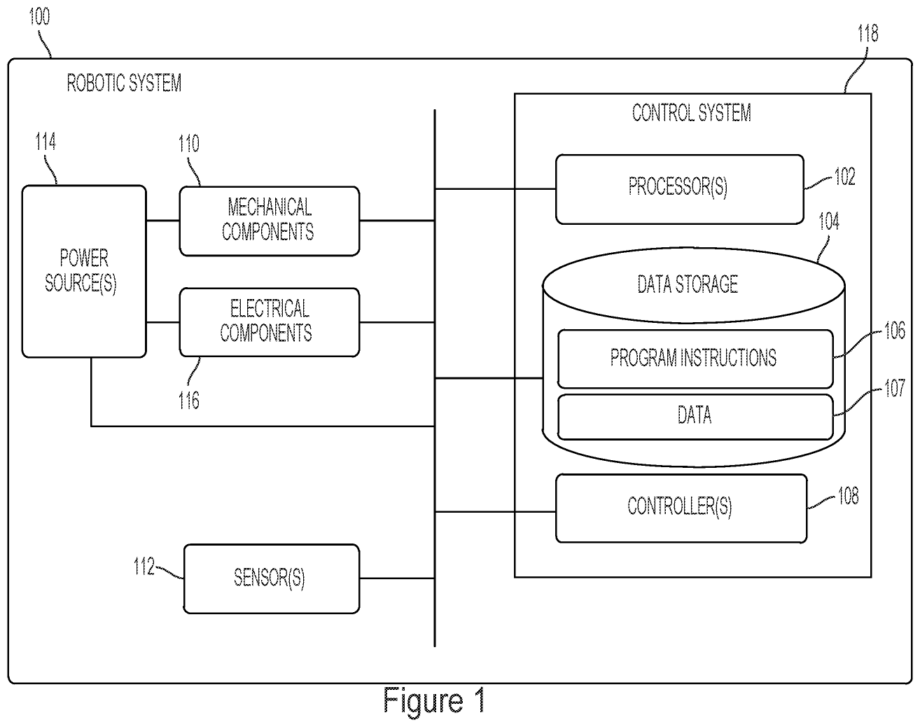

FIG. 1 illustrates an example configuration of a robotic system that may be used in connection with the implementations described herein. Robotic system 100 may be configured to operate autonomously, semi-autonomously, or using directions provided by user(s). Robotic system 100 may be implemented in various forms, such as a robotic arm, industrial robot, or some other arrangement. Some example implementations involve a robotic system 100 engineered to be low cost at scale and designed to support a variety of tasks. Robotic system 100 may be designed to be capable of operating around people. Robotic system 100 may also be optimized for machine learning. Throughout this description, robotic system 100 may also be referred to as a robot, robotic device, or mobile robot, among other designations.

As shown in FIG. 1, robotic system 100 may include processor(s) 102, data storage 104, and controller(s) 108, which together may be part of control system 118. Robotic system 100 may also include sensor(s) 112, power source(s) 114, mechanical components 110, and electrical components 116. Nonetheless, robotic system 100 is shown for illustrative purposes, and may include more or fewer components. The various components of robotic system 100 may be connected in any manner, including wired or wireless connections. Further, in some examples, components of robotic system 100 may be distributed among multiple physical entities rather than a single physical entity. Other example illustrations of robotic system 100 may exist as well.

Processor(s) 102 may operate as one or more general-purpose hardware processors or special purpose hardware processors (e.g., digital signal processors, application specific integrated circuits, etc.). Processor(s) 102 may be configured to execute computer-readable program instructions 106, and manipulate data 107, both of which are stored in data storage 104. Processor(s) 102 may also directly or indirectly interact with other components of robotic system 100, such as sensor(s) 112, power source(s) 114, mechanical components 110, or electrical components 116.

Data storage 104 may be one or more types of hardware memory. For example, data storage 104 may include or take the form of one or more computer-readable storage media that can be read or accessed by processor(s) 102. The one or more computer-readable storage media can include volatile or non-volatile storage components, such as optical, magnetic, organic, or another type of memory or storage, which can be integrated in whole or in part with processor(s) 102. In some implementations, data storage 104 can be a single physical device. In other implementations, data storage 104 can be implemented using two or more physical devices, which may communicate with one another via wired or wireless communication. As noted previously, data storage 104 may include the computer-readable program instructions 106 and data 107. Data 107 may be any type of data, such as configuration data, sensor data, or diagnostic data, among other possibilities.

Controller 108 may include one or more electrical circuits, units of digital logic, computer chips, or microprocessors that are configured to (perhaps among other tasks), interface between any combination of mechanical components 110, sensor(s) 112, power source(s) 114, electrical components 116, control system 118, or a user of robotic system 100. In some implementations, controller 108 may be a purpose-built embedded device for performing specific operations with one or more subsystems of the robotic system 100.

Control system 118 may monitor and physically change the operating conditions of robotic system 100. In doing so, control system 118 may serve as a link between portions of robotic system 100, such as between mechanical components 110 or electrical components 116. In some instances, control system 118 may serve as an interface between robotic system 100 and another computing device. Further, control system 118 may serve as an interface between robotic system 100 and a user. In some instances, control system 118 may include various components for communicating with robotic system 100, including a joystick, buttons, or ports, etc. The example interfaces and communications noted above may be implemented via a wired or wireless connection, or both. Control system 118 may perform other operations for robotic system 100 as well.

During operation, control system 118 may communicate with other systems of robotic system 100 via wired or wireless connections, and may further be configured to communicate with one or more users of the robot. As one possible illustration, control system 118 may receive an input (e.g., from a user or from another robot) indicating an instruction to perform a requested task, such as to pick up and move an object from one location to another location. Based on this input, control system 118 may perform operations to cause the robotic system 100 to make a sequence of movements to perform the requested task. As another illustration, a control system may receive an input indicating an instruction to move to a requested location. In response, control system 118 (perhaps with the assistance of other components or systems) may determine a direction and speed to move robotic system 100 through an environment en route to the requested location.

Operations of control system 118 may be carried out by processor(s) 102.

Alternatively, these operations may be carried out by controller(s) 108, or a combination of processor(s) 102 and controller(s) 108. In some implementations, control system 118 may partially or wholly reside on a device other than robotic system 100, and therefore may at least in part control robotic system 100 remotely.

Mechanical components 110 represent hardware of robotic system 100 that may enable robotic system 100 to perform physical operations. As a few examples, robotic system 100 may include one or more physical members, such as an arm, an end effector, a perception housing, a mast, a midsection, a base, and wheels. The physical members or other parts of robotic system 100 may further include actuators arranged to move the physical members in relation to one another. Robotic system 100 may also include one or more structured bodies for housing control system 118 or other components, and may further include other types of mechanical components. The particular mechanical components 110 used in a given robot may vary based on the design of the robot, and may also be based on the operations or tasks the robot may be configured to perform.

In some examples, mechanical components 110 may include one or more removable components. Robotic system 100 may be configured to add or remove such removable components, which may involve assistance from a user or another robot. For example, robotic system 100 may be configured with removable end effectors or digits that can be replaced or changed as needed or desired. In some implementations, robotic system 100 may include one or more removable or replaceable battery units, control systems, power systems, bumpers, or sensors. Other types of removable components may be included within some implementations.

Robotic system 100 may include sensor(s) 112 arranged to sense aspects of robotic system 100. Sensor(s) 112 may include one or more force sensors, torque sensors, velocity sensors, acceleration sensors, position sensors, proximity sensors, motion sensors, location sensors, load sensors, temperature sensors, touch sensors, depth sensors, ultrasonic range sensors, infrared sensors, object sensors, or cameras, among other possibilities. Within some examples, robotic system 100 may be configured to receive sensor data from sensors that are physically separated from the robot (e.g., sensors that are positioned on other robots or located within the environment in which the robot is operating).

Sensor(s) 112 may provide sensor data to processor(s) 102 (perhaps by way of data 107) to allow for interaction of robotic system 100 with its environment, as well as monitoring of the operation of robotic system 100. The sensor data may be used in evaluation of various factors for activation, movement, and deactivation of mechanical components 110 and electrical components 116 by control system 118. For example, sensor(s) 112 may capture data corresponding to the terrain of the environment or location of nearby objects, which may assist with environment recognition and navigation.

In some examples, sensor(s) 112 may include RADAR (e.g., for long-range object detection, distance determination, or speed determination), LIDAR (e.g., for short-range object detection, distance determination, or speed determination), SONAR (e.g., for underwater object detection, distance determination, or speed determination), VICON® (e.g., for motion capture), one or more cameras (e.g., stereoscopic cameras for 3D vision), a global positioning system (GPS) transceiver, or other sensors for capturing information of the environment in which robotic system 100 is operating. Sensor(s) 112 may monitor the environment in real time, and detect obstacles, elements of the terrain, weather conditions, temperature, or other aspects of the environment. In another example, sensor(s) 112 may capture data corresponding to one or more characteristics of a target or identified object, such as a size, shape, profile, structure, or orientation of the object.

Further, robotic system 100 may include sensor(s) 112 configured to receive information indicative of the state of robotic system 100, including sensor(s) 112 that may monitor the state of the various components of robotic system 100. Sensor(s) 112 may measure activity of systems of robotic system 100 and receive information based on the operation of the various features of robotic system 100, such as the operation of an extendable arm, an end effector, other mechanical or electrical features of robotic system 100. The data provided by sensor(s) 112 may enable control system 118 to determine errors in operation as well as monitor overall operation of components of robotic system 100.

As an example, robotic system 100 may use force/torque sensors to measure load on various components of robotic system 100. In some implementations, robotic system 100 may include one or more force/torque sensors on an arm or end effector to measure the load on the actuators that move one or more members of the arm or end effector. In some examples, the robotic system 100 may include a force/torque sensor at or near the wrist or end effector, but not at or near other joints of a robotic arm. In further examples, robotic system 100 may use one or more position sensors to sense the position of the actuators of the robotic system. For instance, such position sensors may sense states of extension, retraction, positioning, or rotation of the actuators on an arm or end effector.

As another example, sensor(s) 112 may include one or more velocity or acceleration sensors. For instance, sensor(s) 112 may include an inertial measurement unit (IMU). The IMU may sense velocity and acceleration in the world frame, with respect to the gravity vector. The velocity and acceleration sensed by the IMU may then be translated to that of robotic system 100 based on the location of the IMU in robotic system 100 and the kinematics of robotic system 100.

Robotic system 100 may include other types of sensors not explicitly discussed herein. Additionally or alternatively, the robotic system may use particular sensors for purposes not enumerated herein.

Robotic system 100 may also include one or more power source(s) 114 configured to supply power to various components of robotic system 100. Among other possible power systems, robotic system 100 may include a hydraulic system, electrical system, batteries, or other types of power systems. As an example illustration, robotic system 100 may include one or more batteries configured to provide charge to components of robotic system 100. Some of mechanical components 110 or electrical components 116 may each connect to a different power source, may be powered by the same power source, or be powered by multiple power sources.

Any type of power source may be used to power robotic system 100, such as electrical power or a gasoline engine. Additionally or alternatively, robotic system 100 may include a hydraulic system configured to provide power to mechanical components 110 using fluid power. Components of robotic system 100 may operate based on hydraulic fluid being transmitted throughout the hydraulic system to various hydraulic motors and hydraulic cylinders, for example. The hydraulic system may transfer hydraulic power by way of pressurized hydraulic fluid through tubes, flexible hoses, or other links between components of robotic system 100. Power source(s) 114 may charge using various types of charging, such as wired connections to an outside power source, wireless charging, combustion, or other examples.

Electrical components 116 may include various mechanisms capable of processing, transferring, or providing electrical charge or electric signals. Among possible examples, electrical components 116 may include electrical wires, circuitry, or wireless communication transmitters and receivers to enable operations of robotic system 100. Electrical components 116 may interwork with mechanical components 110 to enable robotic system 100 to perform various operations. Electrical components 116 may be configured to provide power from power source(s) 114 to the various mechanical components 110, for example. Further, robotic system 100 may include electric motors. Other examples of electrical components 116 may exist as well.

Robotic system 100 may include a body, which may connect to or house appendages and components of the robotic system. As such, the structure of the body may vary within examples and may further depend on particular operations that a given robot may have been designed to perform. For example, a robot developed to carry heavy loads may have a wide body that enables placement of the load. Similarly, a robot designed to operate in tight spaces may have a relatively tall, narrow body. Further, the body or the other components may be developed using various types of materials, such as metals or plastics. Within other examples, a robot may have a body with a different structure or made of various types of materials.

The body or the other components may include or carry sensor(s) 112. These sensors may be positioned in various locations on the robotic system 100, such as on a body, a perception housing, or an end effector, among other examples.

Robotic system 100 may be configured to carry a load, such as a type of cargo that is to be transported. In some examples, the load may be placed by the robotic system 100 into a bin or other container attached to the robotic system 100. The load may also represent external batteries or other types of power sources (e.g., solar panels) that the robotic system 100 may utilize. Carrying the load represents one example use for which the robotic system 100 may be configured, but the robotic system 100 may be configured to perform other operations as well.

As noted above, robotic system 100 may include various types of appendages, wheels, end effectors, gripping devices and so on. In some examples, robotic system 100 may include a mobile base with wheels, treads, or some other form of locomotion. Additionally, robotic system 100 may include a robotic arm or some other form of robotic manipulator. In the case of a mobile base, the base may be considered as one of mechanical components 110 and may include wheels, powered by one or more of actuators, which allow for mobility of a robotic arm in addition to the rest of the body.

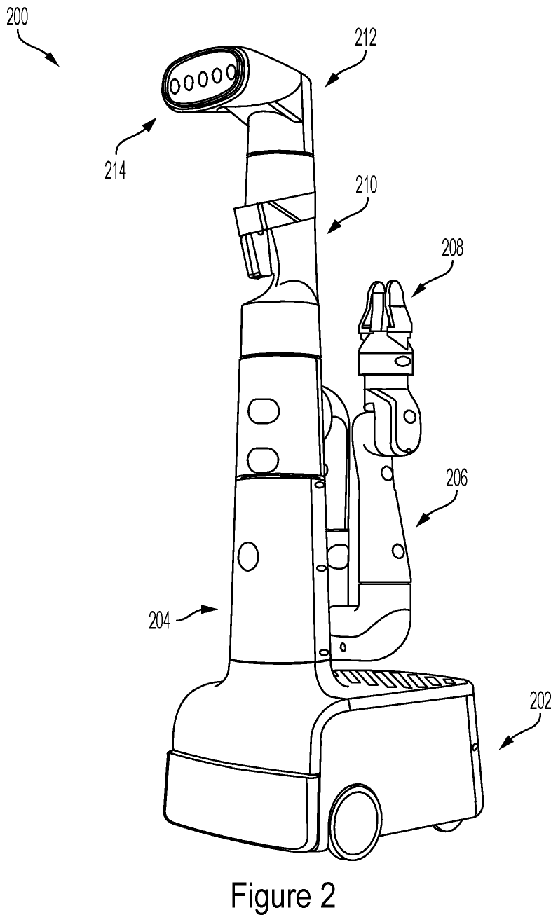

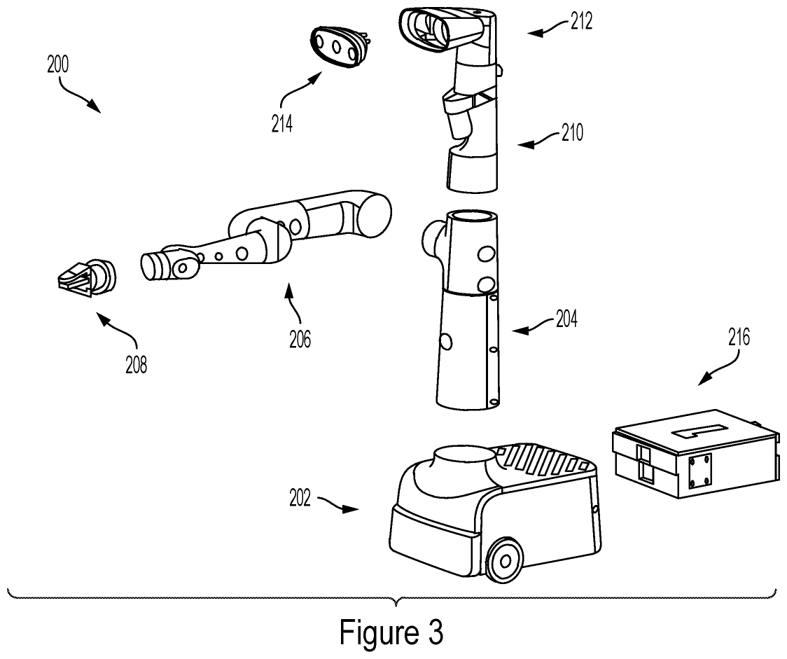

FIG. 2 illustrates a mobile robot, in accordance with example embodiments. FIG. 3 illustrates an exploded view of the mobile robot, in accordance with example embodiments. More specifically, a robot 200 may include a mobile base 202, a midsection 204, an arm 206, an end-of-arm system (EOAS) 208, a mast 210, a perception housing 212, and a perception suite 214. The robot 200 may also include a compute box 216 stored within mobile base 202.

The mobile base 202 includes two drive wheels positioned at a front end of the robot 200 in order to provide locomotion to robot 200. The mobile base 202 also includes additional casters (not shown) to facilitate motion of the mobile base 202 over a ground surface. The mobile base 202 may have a modular architecture that allows compute box 216 to be easily removed. Compute box 216 may serve as a removable control system for robot 200 (rather than a mechanically integrated control system). After removing external shells, the compute box 216 can be easily removed, tested, debugged, and/or replaced. Modularity within compute box 216 may additionally allow the control and perception systems to be independently upgraded. Physical modules inside compute box 216 may also be arranged to minimize cables. The boards inside may interlock in a structure to expose connectors where they are needed externally instead of running cables internal to the compute box 216. The mobile base 202 may also be designed to allow for additional modularity. For example, the mobile base 202 may also be designed so that a power system, a battery, and/or external bumpers can all be easily removed and/or replaced.

The midsection 204 may be attached to the mobile base 202 at a front end of the mobile base 202. The midsection 204 includes a mounting column which is fixed to the mobile base 202. The midsection 204 additionally includes a rotational joint for arm 206. More specifically, the midsection 204 includes the first two degrees of freedom for arm 206 (a shoulder yaw J0 joint and a shoulder pitch J1 joint). The mounting column and the shoulder yaw J0 joint may form a portion of a stacked tower at the front of mobile base 202. The mounting column and the shoulder yaw J0 joint may be coaxial. The length of the mounting column of midsection 204 may be chosen to provide the arm 206 with sufficient height to perform manipulation tasks at commonly encountered height levels (e.g., coffee table top and countertop levels). The length of the mounting column of midsection 204 may also allow the shoulder pitch J1 joint to rotate the arm 206 over the mobile base 202 without contacting the mobile base 202.

The arm 206 may be a 7DOF robotic arm when connected to the midsection 204. As noted, the first two DOFs of the arm 206 may be included in the midsection 204. The remaining five DOFs may be included in a standalone section of the arm 206 as illustrated in FIGS. 2 and 3. The arm 206 may be made up of plastic monolithic link structures. Inside the arm 206 may be housed standalone actuator modules, local motor drivers, and thru bore cabling. Exemplary joint types, ROMs, link lengths, and joint offsets of the arm 206 are described in more detail below.

The EOAS 208 may be an end effector at the end of arm 206. EOAS 208 may allow the robot 200 to manipulate objects in the environment. As shown in FIGS. 2 and 3, EOAS 208 may be a gripper, such as an underactuated pinch gripper. The gripper may include one or more contact sensors such as force/torque sensors and/or non-contact sensors such as one or more cameras to facilitate object detection and gripper control. EOAS 208 may also be a different type of gripper such as a suction gripper or a different type of tool such as a drill or a brush. EOAS 208 may also be swappable or include swappable components such as gripper digits. In some examples, the EOAS 208 may provide means for the robot 200 to sanitize surfaces.

The mast 210 may be a relatively long, narrow component between the shoulder yaw J0 joint for arm 206 and perception housing 212. The mast 210 may be part of the stacked tower at the front of mobile base 202. The mast 210 may be fixed relative to the mobile base 202. The mast 210 may be coaxial with the midsection 204. The length of the mast 210 may facilitate perception by perception suite 214 of objects being manipulated by EOAS 208. The mast 210 may have a length such that when the shoulder pitch J1 joint is rotated vertical up, a topmost point of a bicep of the arm 206 is approximately aligned with a top of the mast 210. The length of the mast 210 may then be sufficient to prevent a collision between the perception housing 212 and the arm 206 when the shoulder pitch J1 joint is rotated vertical up.

As shown in FIGS. 2 and 3, the mast 210 may include a 3D lidar sensor configured to collect depth information about the environment. The 3D lidar sensor may be coupled to a carved out portion of the mast 210 and fixed at a downward angle. The lidar position may be optimized for localization, navigation, and for front cliff detection.

The perception housing 212 may include at least one sensor making up perception suite 214. The perception housing 212 may be connected to a pan/tilt control to allow for reorienting of the perception housing 212 (e.g., to view objects being manipulated by EOAS 208). The perception housing 212 may be a part of the stacked tower fixed to the mobile base 202. A rear portion of the perception housing 212 may be coaxial with the mast 210.

The perception suite 214 may include a suite of sensors configured to collect sensor data representative of the environment of the robot 200. The perception suite 214 may include an infrared (IR)-assisted stereo depth sensor. The perception suite 214 may additionally include a wide-angled red-green-blue (RGB) camera for human-robot interaction and context information. The perception suite 214 may additionally include a high resolution RGB camera for object classification. A face light ring surrounding the perception suite 214 may also be included for improved human-robot interaction and scene illumination.

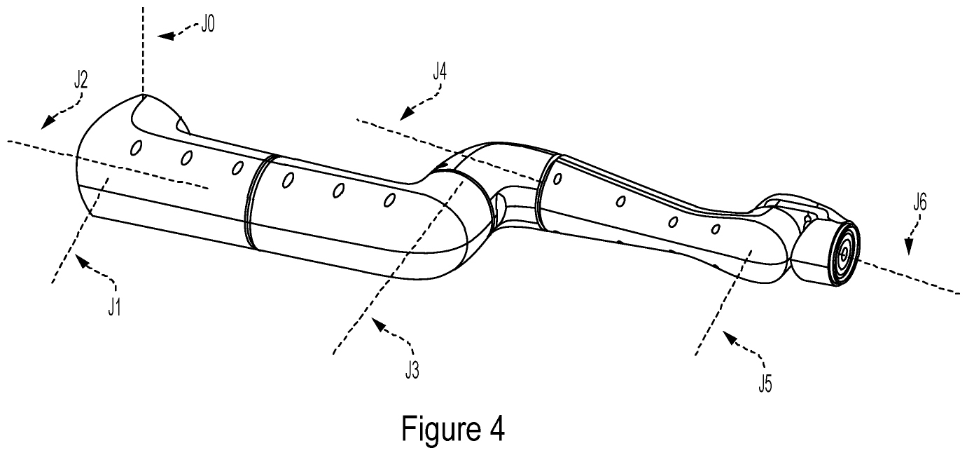

FIG. 4 illustrates a robotic arm, in accordance with example embodiments. The robotic arm includes 7 DOFs: a shoulder yaw J0 joint, a shoulder pitch J1 joint, a bicep roll J2 joint, an elbow pitch J3 joint, a forearm roll J4 joint, a wrist pitch J5 joint, and wrist roll J6 joint. Each of the joints may be coupled to one or more actuators. The actuators coupled to the joints may be operable to cause movement of links down the kinematic chain (as well as any end effector attached to the robot arm).

The shoulder yaw J0 joint allows the robot arm to rotate toward the front and toward the back of the robot. One beneficial use of this motion is to allow the robot to pick up an object in front of the robot and quickly place the object on the rear section of the robot (as well as the reverse motion). Another beneficial use of this motion is to quickly move the robot arm from a stowed configuration behind the robot to an active position in front of the robot (as well as the reverse motion).

The shoulder pitch J1 joint allows the robot to lift the robot arm (e.g., so that the bicep is up to perception suite level on the robot) and to lower the robot arm (e.g., so that the bicep is just above the mobile base). This motion is beneficial to allow the robot to efficiently perform manipulation operations (e.g., top grasps and side grasps) at different target height levels in the environment. For instance, the shoulder pitch J1 joint may be rotated to a vertical up position to allow the robot to easily manipulate objects on a table in the environment. The shoulder pitch J1 joint may be rotated to a vertical down position to allow the robot to easily manipulate objects on a ground surface in the environment.

The bicep roll J2 joint allows the robot to rotate the bicep to move the elbow and forearm relative to the bicep. This motion may be particularly beneficial for facilitating a clear view of the EOAS by the robot's perception suite. By rotating the bicep roll J2 joint, the robot may kick out the elbow and forearm to improve line of sight to an object held in a gripper of the robot.

Moving down the kinematic chain, alternating pitch and roll joints (a shoulder pitch J1 joint, a bicep roll J2 joint, an elbow pitch J3 joint, a forearm roll J4 joint, a wrist pitch J5 joint, and wrist roll J6 joint) are provided to improve the manipulability of the robotic arm. The axes of the wrist pitch J5 joint, the wrist roll J6 joint, and the forearm roll J4 joint are intersecting for reduced arm motion to reorient objects. The wrist roll J6 point is provided instead of two pitch joints in the wrist in order to improve object rotation.

In some examples, a robotic arm such as the one illustrated in FIG. 4 may be capable of operating in a teach mode. In particular, teach mode may be an operating mode of the robotic arm that allows a user to physically interact with and guide robotic arm towards carrying out and recording various movements. In a teaching mode, an external force is applied (e.g., by the user) to the robotic arm based on a teaching input that is intended to teach the robot regarding how to carry out a specific task. The robotic arm may thus obtain data regarding how to carry out the specific task based on instructions and guidance from the user. Such data may relate to a plurality of configurations of mechanical components, joint position data, velocity data, acceleration data, torque data, force data, and power data, among other possibilities.

During teach mode the user may grasp onto the EOAS or wrist in some examples or onto any part of the robotic arm in other examples and provide an external force by physically moving the robotic arm. In particular, the user may guide the robotic arm towards grasping onto an object and then moving the object from a first location to a second location. As the user guides the robotic arm during teach mode, the robot may obtain and record data related to the movement such that the robotic arm may be configured to independently carry out the task at a future time during independent operation (e.g., when the robotic arm operates independently outside of teach mode). In some examples, external forces may also be applied by other entities in the physical workspace such as by other objects, machines, or robotic systems, among other possibilities.

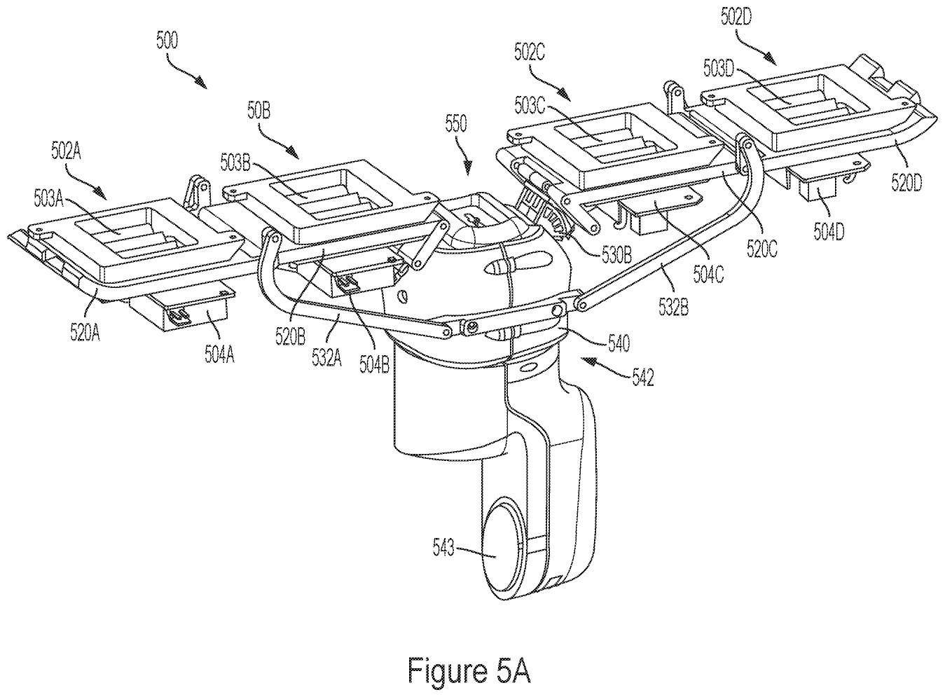

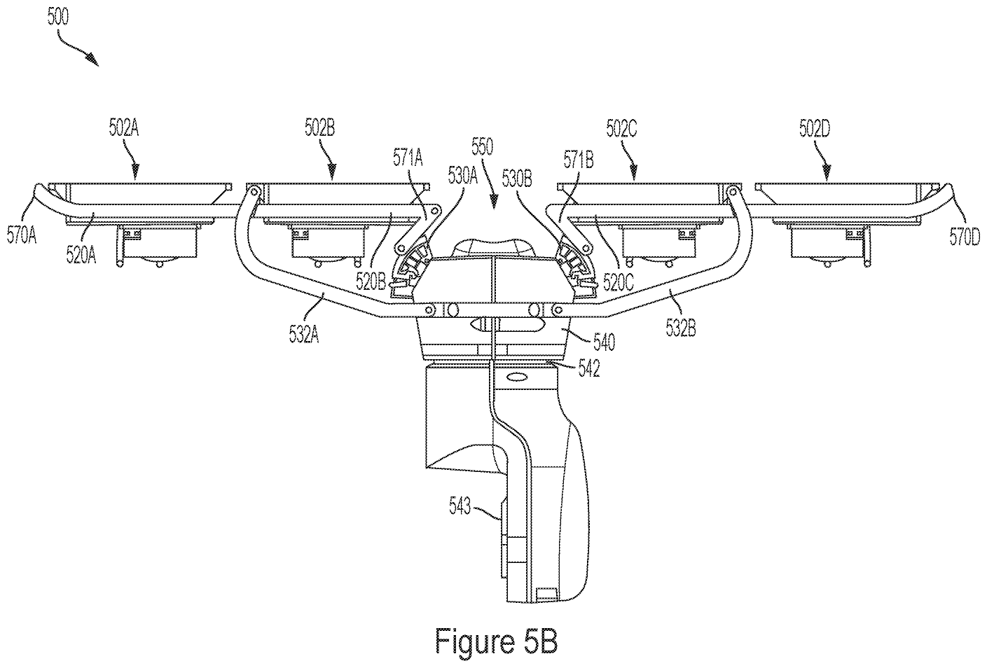

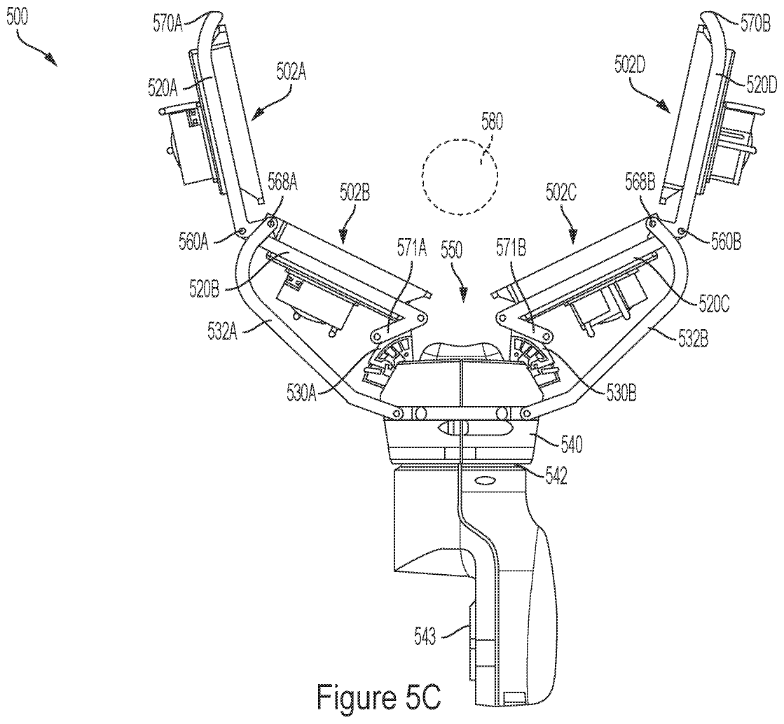

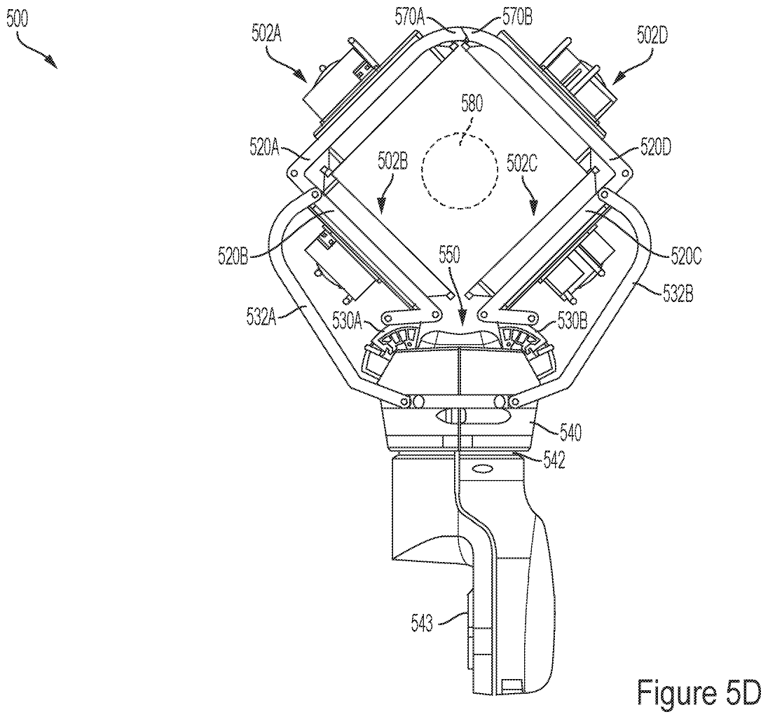

FIGS. 5A-5D depict an end effector 500. The end effector 500 includes a plurality of UV light modules 502A-D. In some examples, the plurality of UV light modules 502A-D may be considered an array of UV light modules, and specifically a foldable array of UV light modules. As illustrated in FIGS. 5A-5D, the plurality of UV light modules 502A-D includes a first UV light module 502A, a second UV light module 502B, a third UV light module 502C, and a fourth UV light module 502D. As such, in some examples, the plurality of light modules 502A-D may include four UV light modules. In other examples, the plurality of UV light modules may include two, three, or more than four UV light modules. FIG. 5A and FIG. 5B depicts the plurality of UV light modules 502A-D in a first alignment, in one example embodiment. Figure depicts the plurality of UV light modules 502A-D in a second alignment, in one example embodiment. FIG. 5D depicts the plurality of UV light modules 502A-D in a third alignment, in one example embodiment.

A UV light module may be configured to emit UV radiation in order to sanitize a surface. In order to operate, each of the UV light modules 502A-D may include one or more UV lamps 503A-D and a transformer 504A-D. The UV lamps 503A-D are configured to emit UV radiation. In order to emit UV radiation, and specifically UV-C radiation, each of the UV lamps 503A-D may require a high voltage source. Moreover, in order to reduce any voltage drop and for other safety considerations, it may be preferable to reduce a distance between the high voltage source(s) and the UV light modules 502A-D. In some examples, the transformers 504A-D may be considered a high-voltage transformer and be coupled to a back side of each of the UV lamps 503A-D. In other examples, there may be a single high voltage transformer that is coupled to each of the UV lamps 503A-D of the UV light modules 502A-D.

Among other possibilities, the UV light modules may include commercially available UV lamps configured to sanitize surfaces exposed to UV light, such as the Care222® Far UV-C Excimer lamps by Ushio. In order to effectively sanitize a surface, a UV light module may need to maintain a certain proximity to the target surface for a given amount of time. The proximity to the surface and amount of time required to sanitize a surface is dependent upon a variety of factors, including specifications of the UV lamps used. Moreover, the closer to the surface, the less time may be necessary to effectively sanitize a surface. Additionally, being able to move UV lamps into specific positions to directly expose surfaces requiring sanitization may be more effective at sanitizing the surface.

The end effector 500 further includes a plurality of array segments 520A-D. As illustrated in FIGS. 5A-5D, the plurality of array segments 520A-D includes a first array segment 520A, a second array segment 520B, a third array segment 520C, and a fourth array segment 520D. In other example arrangements or configurations, there may be more or less than four array segments. Moreover, each UV light module of the plurality of light modules 502A-D may be coupled to a different array segment of the plurality of array segments 520A-D. For example, as illustrated in FIGS. 5A-5D: the first UV light module 502A is coupled to the first array segment 520A; the second UV light module 502B is coupled to the second array segment 520B; the third UV light module 502C is coupled to the third array segment 520C: and, the fourth UV light module 502D is coupled to the fourth array segment 520D. In some examples, the UV lamps 503A-D may be coupled on one side of each of the array segments 520A-D while the transformers 504A-D are coupled on another side of each of the array segments 520A-D, respectively.

The end effector 500 also includes a first articulating member 530A and a second articulating member 530B. The articulating members 530A-B may be coupled to and included as part of a housing 540 of the end effector 500. The housing 540 of the end effector 500 may include or be coupled to a joint 542. In some examples, the joint 542 may be where the end effector 500 couples to a part of a robotic system, such as coupling to a robotic arm, for example. The joint 542 may be a joint of a robotic arm, such as the wrist roll J6 joint of the robotic arm of FIG. 4 as described above. In some examples, the housing 540 may also be considered to include a second joint 543. The second joint 543 may be another joint of a robotic arm, such as the wrist pitch J5 joint. In some examples, the end effector 500 may not include the joint 542 and the second joint 543. In other examples, the joint 543 may couple to another portion of a robotic device.

The end effector 500 further includes a sensor 550. The sensor 550, among other possibilities, may determine a proximity of the end effector 500, or portions thereof, to a surface. In further examples, the sensor 550 may be arranged at a different position and/or orientation proximate to the end effector 500 than specifically illustrated here.

The articulating members 530A-B may be powered and configured to cause at least a portion of the plurality of array segments 520A-D to move relative to one another. The relative motions of the plurality of array segments 520A-D may result or cause relative movement of the plurality of UV light modules 502A-D. The relative movement may depend on additional features of the end effector 500, such as a set of first connection points 560A-B and a set of second connection points 568A-B. The set of first connection points 560A-B may provide for a rotatable connection between array segments. For example, the first connection point 560A may be a point where the first array segment 520A couples to the second array segment 520B. The set of second connections points 568A-B may provide for a rotatable connection between support guides 532A-B and array segments 520A-D. More particularly, the support guides 532A-B may couple between outer array segments, that is the first array segment 520A and the fourth array segment 520D, and the housing 540. Specifically, the support guide 532A, for example, couples to the first array segment 520A at the second connection point 568A. The support guides 532A-B may be configured to guide movement of the array segments 520A-D. In some examples, the support guides 532A-B may be configured to control the movement of the array segments 520A-D and the UV-light modules 502A-D relative to one another. The support guides 532A-B have a curved portion or shape as depicted such that the support guides 532A-B avoid conflicting with other portions of the end effector 500. For example the curved shape of the support guides 532A-B may avoid contacting the transformers 504A-D of the UV modules 502A-D, specifically the inner UV modules 502B-C and transformers 504B-C. In other examples, the support guides 532A-B may mechanically couple and support movement of the outer array segments (for example, array segments 520A and 520D). In yet other examples, the support guides 532A-B may be straight components.

The first array segment 520A and the fourth array segment 520D, together the outer array segments in the embodiment depicted in FIGS. 5A-5D, may further include distal ends 570A-B. For example, the first array segment 520A may include the distal end 570A while the fourth array segment 520D may include the distal end 570B. In certain arrangements, the distal end 570A may be configured to couple to the distal end 570B.

The second array segment 520B and the third array segment 520C, together the inner array segments in the embodiment depicted in FIGS. 5A-5D, may further include proximate ends 571A-B. For example, the second array segment 520B may include the proximate end 571A while the third array segment 520C may include the proximate end 571B. The proximate end 571A of the second array segment 520B is coupled to the first articulating member 530A. Similarly, the proximate end 571B of the third array segment 520C is coupled to the second articulating member 530B.

With the configuration described and depicted in FIGS. 5A-D, the end effector 500, and particularly the array segments 520A-D and the UV light modules 502A-D may be configured to surround, frame, and/or enclose a surface, such as the non-planar surface 580 provided in FIGS. 5C and 5D.

Reviewing the movement depicted in FIGS. 5A-5D, in FIGS. 5A and 5B the first articulating member 530A is in a first position. Moreover, the second articulating member 530B may also be in a first position. In this arrangement with the articulating members 530A-B in first positions, the plurality of UV light modules may be in a first alignment relative to one another. The first alignment may be considered a planar or linear arrangement. In some examples, the first alignment may be considered approximately planar or approximately linear. In the arrangement shown in FIGS. 5A and 5B, the plurality of UV light modules 502A-D may be arranged such that the plurality of UV light modules 502A-D are configured to emit UV-light in substantially parallel directions relative to one another. This arrangement may be considered a planar or substantially planar arrangement. As shown in FIGS. 5A and 5B, the end effector 500, with the plurality of UV light modules 502A-D arranged substantially linear relative one another, may be configured to sanitize a planar or substantially planar surface using UV light.

Continuing, in FIG. 5C the first and second articulating members 530A-B may have moved such that the plurality of array segments 520A-D and the plurality of UV light modules 502A-D are in a second alignment relative to one another. For example, the movement of the articulating member 530A causes movement of the second array segment 520B and second UV light module 502B and movement of the first array segment 520A and the first UV light module 502A. Thus, the movement of the articulating member 530A causes the relative alignment of the first UV light module 502A to change relative to the second UV light module 502B. In the alignment depicted in FIG. 5C, the UV light modules 502A-D are arranged relative to one another such that the UV light modules 502A-D emit UV light in substantially non-planar directions relative to one another. This arrangement may directly expose more of the surface 580 to sanitizing UV light than the arrangement in FIGS. 5A-5B would otherwise.

Thus, in some embodiments, the first articulating member 530A may cause movement and a change to the relative alignment of the first and second UV light modules 502A-B, even though the first articulating member 530A is only directly coupled at a single location, the proximate end 571A of the second array segment 520B.

Continuing to FIG. 5D, the first and second articulating members 530A-B have moved to yet another position, which may be considered a third position. In this position, the plurality of UV light modules 502A-D may surround the surface 580. As depicted, the distal end 570A is coupled to the distal end 570B such that the end effector 500 is surrounding at least a portion of the non-planar surface 580.

In order to effectively and predictably sanitize a surface, it may be useful to keep each of the plurality of UV light modules 502A-D approximately the same distance from the surface being sanitized. As such, in some examples, the plurality of UV light modules 502A-D are equidistant from a surface, such as the surface 580 in FIG. 5D. In other examples, the plurality of UV light modules 502A-D are equidistant from an axis when the UV light modules are arranged to entirely surround a surface.

While the Figures illustrate the first and second articulating members 530A-B moving together, it should be understood that in certain example embodiments the two articulating members may be able to move separate from one another. In other examples, only one articulating member and half the UV light modules shown in FIGS. 5A-5D may be provided. Moreover, it should be recognized that what was described as the “first position,” “second position,” and “third position,” in FIGS. 5A-5D are relative and any of the positions could be the second, third, or another iterative position relative to a reference position.

In some examples, the relative arrangement of the plurality of UV light modules 502A-D is based on a determination of the shape and/or features of a surface to be sanitized. For example, the surface may be an edge of a desk or table or similar object and in such a case it may be determined to move the first articulating member 530A to one position, but to leave the second articulating member 530B in a reference position, for example. A variety of arrangements and examples will be apparent to a person of skill in the art in view of the disclosure.

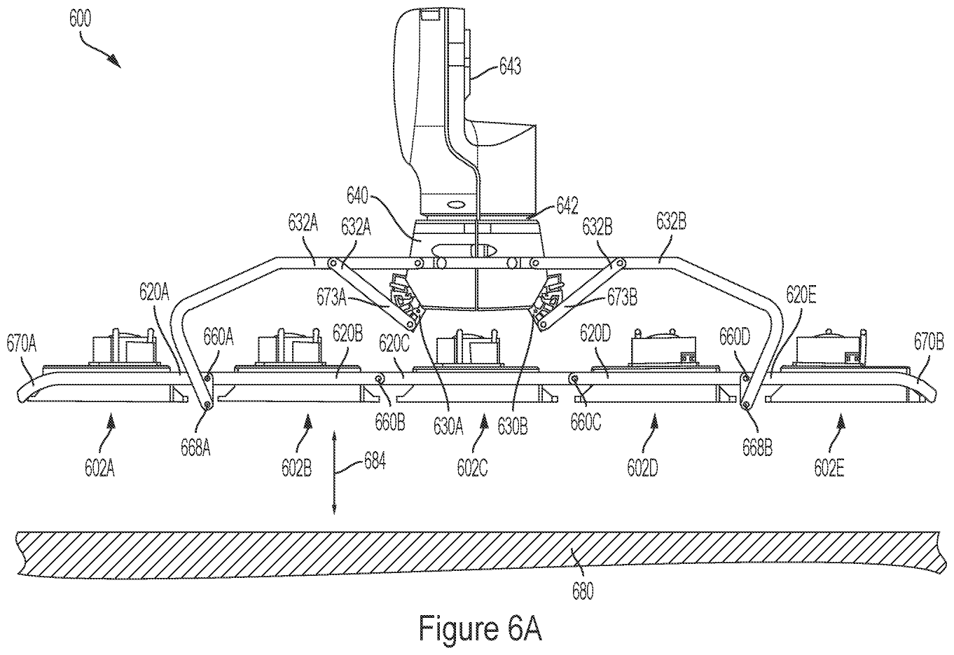

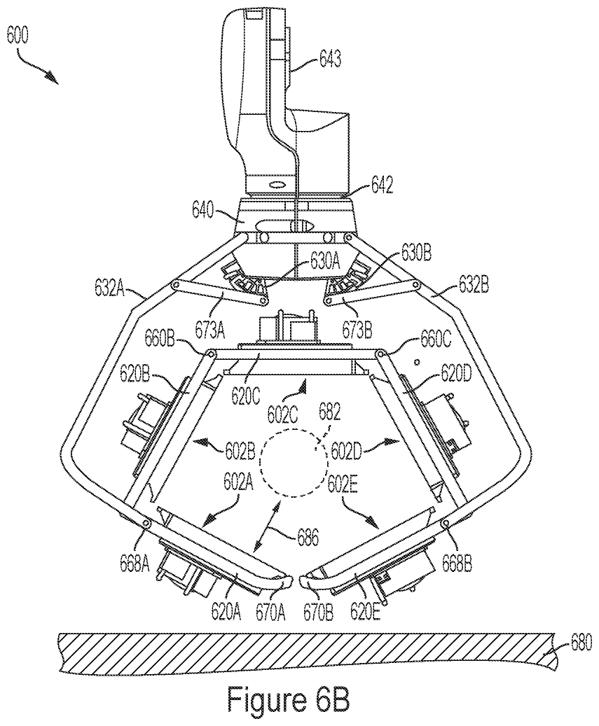

FIGS. 6A-6B depict an end effector 600. The end effector 600 may include similar features with similar function as those described in relation to the end effector 500. The similar features may have similar reference numbers as those of end effector 500. The end effector 600 includes a plurality of UV light modules 601A-E, a plurality of array segments 620A-E, a first and a second articulating member 630A-B, a plurality of support guides 632A-B, and a housing 640. Each of the plurality of UV light modules 602A-E may be coupled to one of the plurality of array segments 620A-E. The end effector 600 also includes a plurality of first connection points 660A-D and a plurality of second connection points 668A-B. As shown, the end effector 600 may include five array segments 620A-E and five UV light modules 602A-E.

As shown in FIGS. 6A-B, each of the support guides 632A-B may include a proximate portion 673A-B, respectively. In some example configurations, the support guides 632A-B may be considered articulating arms and part of the articulating members 630A-B. The proximate portions 673A-B of the support guides 632A-B may be coupled to the first and second articulating members 630A-B, respectively. Moreover, the support guides 632A-B may be coupled to outer array segments, which in FIGS. 6A-B include the first array segment 620A and the fifth array segment 620. Particularly, the support guide 632A may be coupled to the first array segment 620A at the second connection point 668A and the support guide 632B may be coupled to the fifth array segment 620E at the second connection point 668B. Within specific examples, the second connection points 668A-B may be located on an extended proximate portion of the out array segments 620A and 620E. The extended proximate portion may be opposite distal portions 670A and 670B of the outer array segments 620A and 620E, respectively. The extended proximate portions of the outer array segments may extend in the direction that the UV light modules 602A and 602E are configured to emit UV light. This arrangement may mechanically allow for the support guides 632A-B to cause the outer array segments 620A and 620E to move, as described in more detail below.

Regarding FIG. 6A, the first and second articulating member 630A-B may each be in a first position. In some regards, the first position may be a position relative to the housing 640, for example. In some aspects, the first position may be considered an open or a fully open position such that the plurality of UV light modules 602A-E are arranged linearly relative to one another. In this arrangement, the UV light modules 602A-E may be configured to sanitize a substantially or primarily planar surface, such as the surface 680. In order to sanitize the surface 680, the plurality of UV light modules 602A-E may be positioned at a distance 684 from the surface 680.

In order to effectively sanitize the surface 680, it may be important to maintain the distance 684 from the surface 680 for a predetermined amount of time. Moreover, the amount of time necessary to sanitize the surface 680 may be based on the intensity of the UV light modules 602A-E as well as the distance 684. As the distance 684 increases, so does the amount of time that the surface 680 must be exposed to the UV radiation from the UV light modules in order to effectively sanitize the surface. The distance 684 may be determined and/or maintained using sensor data from a sensor within the end effector 600 or a robotic system or device coupled to the end effector 600.

Continuing to FIG. 6B, the end effector 600 may be configured to move at least a portion of the array segments 620A-E, and the UV light modules 602A-E correspondingly, to better expose various features or surfaces of a non-planar surface to UV radiation. For example, the UV light modules 602A-E may be configured to close around or surround a round surface 682. The round surface 682 may be a portion of a railing or door handle, for example. In other examples, a non-planar surface may include an edge, such as an edge of a desk or the edge of a wall that exists in multiple planes such that UV light sanitization may be effective implemented where the UV light modules 602A-E are able to face multiple directions to expose multiple planes or features of a surface with UV light simultaneously.

To facilitate the movement, the articulating members 630A-B may cause the relative motion of the plurality of array segments 620A-E and the plurality of UV light modules 602A-E. It should be understood that this may be accomplished using a variety of configurations of the support guides 632A-B and array segments 620A-E. For example, as provided in the configuration in FIGS. 6A-B, this motion may be completed via the support guides 632A-B. Specifically, the articulating members 630A and 630B may rotate inwards from the first position (FIG. 6A) towards a second position, for example as depicted in FIG. 6B. The rotation of the articulating members 630A-B may mechanically pull the proximate portions 673A-B of the support guides 632A-B causing the support guides 632A-B to rotate relative to the housing 640. The movement of the support guides 632A-B may cause movement of the outer array segments 620A and 620E coupled to the support guides 632A-B. Within examples, after causing at least some of the plurality of array segments 620A-E to move relative to one another, the UV light modules 602A-E may end up being a distance 686 away from the round surface 682. In some examples, the UV light modules 602A-E may be equidistant from an axis or edge of surface in order to effectively and substantially uniformly expose the surface to UV radiation.

Within examples, the movement of one array segment may cause relative movement of at least one other array segment. For example, the movement of the first array segment 620A caused by the first articulating member 630A may cause relative movement of the second array segment 620B. In some examples, this may be considered a folding motion where at least one array segment of the plurality of array segments 620A-E moves relative to another array segment. The folding motion may be configured to match or correspond to features or shape of a surface to be sanitized.

While the plurality of UV light modules 602A-E and array segments 620A-E are configured to move relative to one another, in some examples at least one UV light module and array segment may be configured to not move relative to the housing 640. For example, as depicted in FIGS. 6A and 6B, the third UV light module 602C and the third array segment 620C may remain in the same position relative to the housing 640, despite the movement of the articulating members 630A-B.

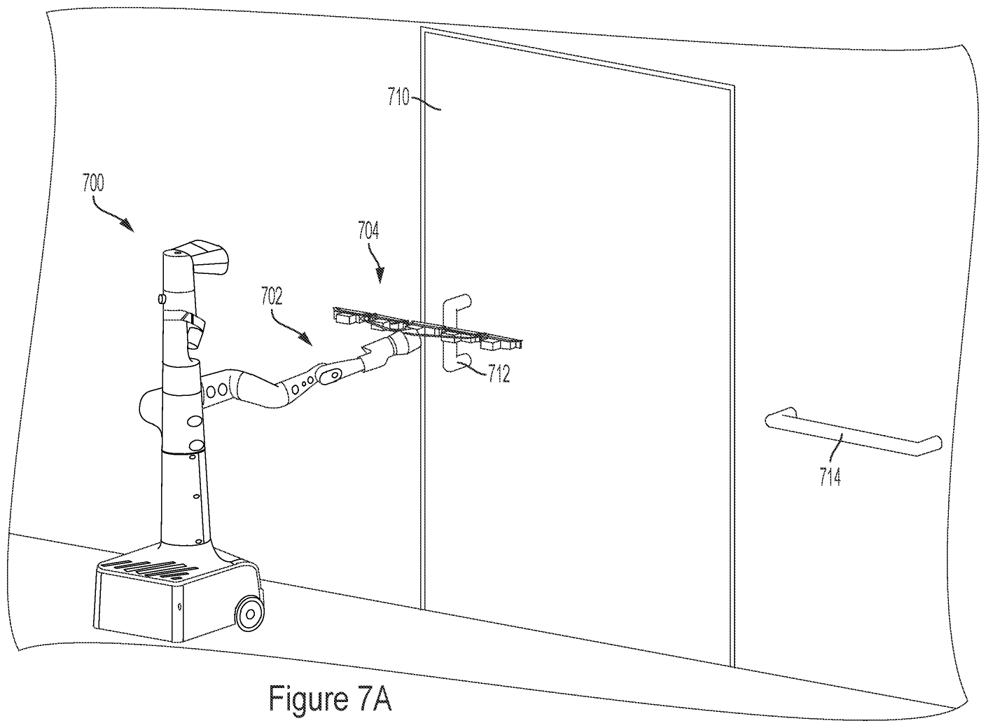

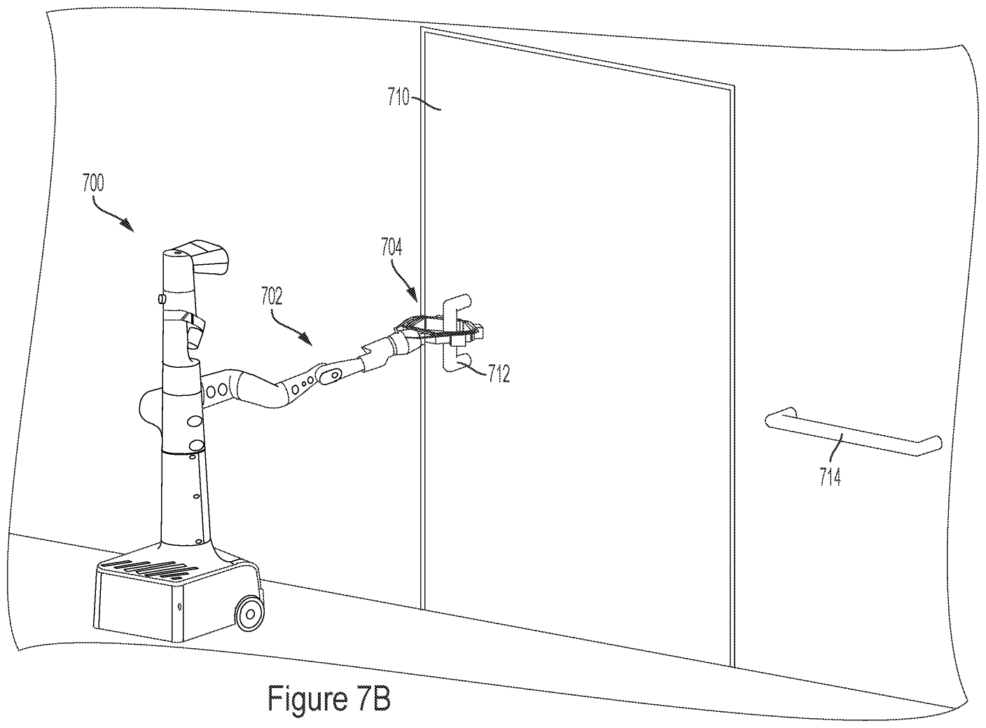

FIGS. 7A-7B depict a mobile robot 700. The mobile robot 700 may be similar in form and function as the robotic system 100 and robot 200 of FIGS. 1-3, respectively. Among a variety of other features, the mobile robot 700 may be configured to sanitize surfaces within an environment using UV light. The mobile robot 700 may include an arm 702, and an end effector 704. The arm 702 may be coupled to a body of the mobile robot 700 as well as the end effector 704. The end effector 704 may be a foldable UV light array end effector, such as the end effector 500 of FIGS. 5A-5D or the end effector 600 of FIGS. 6A-6B. The environment may include a door 710, a door handle 712, and a railing 714, among a variety of other potential surfaces and features. The mobile robot 700 may be equipped with a variety of sensors, including depth sensors and/or visual cameras, that help the robot 700 navigate and determine the shape of surfaces to be sanitized with UV light.

For example, as depicted in FIG. 7A, the mobile robot 700 may approach the door 710. The door 710 may be substantially planar, so the mobile robot 700 may activate a plurality of UV light modules installed in the end effector 704 while the plurality of UV light modules are arranged substantially linear to one another. In this configuration, the mobile robot 700 may wave the end effector 704 including the array of UV light modules across the surface of the door in a manner to effectively sanitize the door. However, the handle 712 may be a non-planar surface with features that are not exposed to the UV light when the plurality of UV light modules are arranged linearly.

As such, the mobile robot 700 may cause the array of UV light modules to fold about a surface, such as the handle 712, as depicted in FIG. 7B. To arrive in this arrangement, at least one articulating member of the end effector 704 may move and cause relative movement of at least one of the plurality of UV light modules relative to one another. As positioned in FIG. 7B, the end effector 704 may surround the door handle 712 and may now distribute UV radiation to the entire surface of the handle 712. The joints of the arm 702 may articulate such that the end effector 704 passes over the various portions and surfaces of the handle 712. In another example, the robot 700 may similarly move to the railing 714 and use at least one articulating member to cause at least one of the plurality of UV light modules to move relative one another in order to emit UV radiation directly onto the round or non-planar surface of the railing 714.

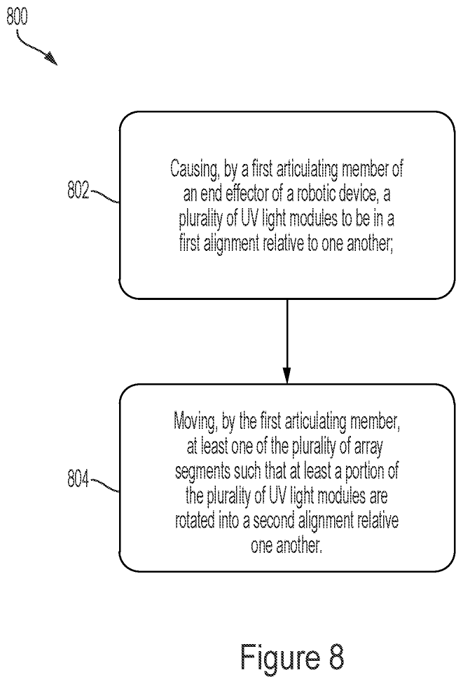

FIG. 8 is a simplified block diagram illustrating a method 800 relating to operating an end effector of a robotic device in order to move UV light modules from a first alignment into a second alignment, according to an example embodiment. It should be understood that example methods, such as method 800, might be carried out by one or more entities, or combinations of entities (i.e., by other computing devices, and/or combinations thereof), without departing from the scope of the invention.

For example, functions of the method 800 may be fully performed by a machine, a human operator, a computing device (or components of a computing device such as one or more processors or controllers), or may be distributed across multiple components of the computing device, across multiple computing devices, and/or across one or more servers. In some examples, the computing device may receive information from input commands initiated by an operator, sensors of the computing device, or may receive information from other computing devices that collect the information. More particularly, functions of the method 800 may be carried out by computing device(s) and/or controller(s) of a mobile robot, or that of a robotic system or network, or a combination thereof.

As shown by block 802, the method 800 includes causing a plurality of UV light modules to be in a first alignment relative to one another. The plurality of UV light modules may be part of an array within an end effector of a robotic device, and a first articulating member of the end effector may cause the UV light modules to be in the first alignment. As used herein, an articulating member refers to a physical component configured to facilitate movement of an array. The articulating member may take on a variety of different shapes and sizes. In some examples, the articulating member may be configured to be actuated by an actuator, such as a linear or rotary actuator. In further examples, the articulating member may be an actuator, such as a linear or rotary actuator, which is directly connected to an array. In yet further examples, the articulating member may include an actuator and one or more intervening components which connect the actuator to the array.

The end effector may be similar and/or include similar features as the end effector 500, the end effector 600, or the end effector 704, as provided in FIGS. 5A-5C, 6A-6B and 7A-7B, respectively. As such, the plurality of UV light modules may be coupled to a plurality of array segments of the end effector. In the first alignment, the UV light modules may be configured to sanitize a first surface that may include a first set of features. In some examples, the first surface may be planar and the UV light modules may be aligned substantially linear to one another.

In some examples, the robotic device may also be configured to sanitize a second surface, and or features of the first surface or second surface that may not be planar. The robotic device may include one or more sensors, and the method 800 may include, determining contours of a surface to be sanitized by one or more of the sensors. Then, based on the contours of the surface, the method 800 may include determining a sanitizing alignment of the plurality of UV light modules. In some examples, the sanitizing alignment may be a second alignment.

As shown by block 804, the method 800 includes moving at least one of the plurality of array segments by the first articulating member such that at least a portion of the plurality of UV light modules are rotated into the second alignment relative to one another. The second alignment may allow the robotic device to more directly and effectively sanitize a surface than the robotic device would have been able to when in the first alignment. For example, in a configuration where the plurality of light modules are substantially planar to one another in the first alignment, a back or side of a surface that extends from another surface may not be exposed to the UV radiation from the plurality of UV light modules in a way that effectively sanitizes the surface.

The method may also include moving at least one other array segment of the plurality of array segments. In some examples, the movement of one array segment might cause motion of another array segment, for example. As such, the movement of the first articulating member may result in the relative movement of more than one array segment. In some examples, the method 800 may also include moving the end effector with the plurality of UV light modules over or across a surface to be sanitized. As such, in some examples, the method 800 may include sanitizing one or more surfaces.

In other embodiments the method 800 may include more or less blocks as well as blocks that carry out various functions described herein. Also, while the blocks are expressed in a specific order herein, other ordering and combinations of the various blocks and steps are considered herein

### III. CONCLUSION

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The above detailed description describes various features and functions of the disclosed systems, devices, and methods with reference to the accompanying figures. In the figures, similar symbols typically identify similar components, unless context dictates otherwise. The example embodiments described herein and in the figures are not meant to be limiting. Other embodiments can be utilized, and other changes can be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

A block that represents a processing of information may correspond to circuitry that can be configured to perform the specific logical functions of a herein-described method or technique. Alternatively or additionally, a block that represents a processing of information may correspond to a module, a segment, or a portion of program code (including related data). The program code may include one or more instructions executable by a processor for implementing specific logical functions or actions in the method or technique. The program code or related data may be stored on any type of computer readable medium such as a storage device including a disk or hard drive or other storage medium.

The computer readable medium may also include non-transitory computer readable media such as computer-readable media that stores data for short periods of time like register memory, processor cache, and random access memory (RAM). The computer readable media may also include non-transitory computer readable media that stores program code or data for longer periods of time, such as secondary or persistent long term storage, like read only memory (ROM), optical or magnetic disks, compact-disc read only memory (CD-ROM), for example. The computer readable media may also be any other volatile or non-volatile storage systems. A computer readable medium may be considered a computer readable storage medium, for example, or a tangible storage device.

Moreover, a block that represents one or more information transmissions may correspond to information transmissions between software or hardware modules in the same physical device. However, other information transmissions may be between software modules or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. An end effector of a robotic device comprising:
a plurality of array segments, wherein each array segment of the plurality of array segments is coupled to at least one other array segment of the plurality of array segments;
a plurality of UV light modules, wherein each UV light module of the plurality of UV light modules is coupled to a different array segment of the plurality of array segments; and
a first articulating member configured to cause at least one of the plurality of array segments to move relative to at least one other array segment.

2. The end effector of claim 1, wherein when the first articulating member is in a first position the plurality of UV light modules are in a first alignment relative to one another, and when the first articulating member is in a second position the plurality of UV light modules are in a second alignment relative to one another.

3. The end effector of claim 2, wherein the first alignment relative to one another comprises the plurality of UV light modules arranged linearly relative to one another.

4. The end effector of claim 2, wherein the second alignment relative to one another comprises the plurality of UV light modules arranged such that the plurality of UV light modules are configured to emit UV-light in substantially non-parallel directions relative to one another.

5. The end effector of claim 2, wherein the second alignment relative to one another comprises the plurality of UV light modules arranged such that they are configured to surround a non-planar surface.

6. The end effector of claim 2, wherein when the first articulating member is in the second position, each UV light module is approximately equidistant from an axis.

7. The end effector of claim 1, wherein the first articulating member is coupled to the plurality of array segments at a single location.

8. The end effector of claim 1, wherein the plurality of array segments is coupled to the first articulating member such that movement of the first articulating member causes at least a portion of the plurality of array segments to move relative to one another.

9. The end effector of claim 1, wherein the plurality of UV light modules comprises two UV light modules.

10. The end effector of claim 1, further comprising:
a second articulating member configured to cause at least a second portion of the plurality of array segments to move relative to one another.

11. The end effector of claim 10, wherein the plurality of UV light modules comprises four UV light modules.

12. The end effector of claim 10, wherein the plurality of array segments comprises a first distal array segment and a second distal array segment, wherein motion of the first distal array segment is caused by the first articulating member and motion of the second distal array segment is caused by the second articulating member, and wherein a distal end of the first distal array segment is configured to couple to a distal end of the second distal array segment when the plurality of UV light modules are arranged such that the plurality of UV light modules surround a non-planar surface.

13. The end effector of claim 1, further comprising:
a proximity sensor configured to determine a distance to a surface.

14. The end effector of claim 1, wherein each of the plurality of UV light modules is configured to emit UV-C radiation.

15. The end effector of claim 1, wherein at least one of the UV light modules is stationary relative to the end effector.

16. A method comprising:
causing, by a first articulating member of an end effector of a robotic device, a plurality of UV light modules to be in a first alignment relative to one another, wherein each of the plurality of UV light modules is coupled to one of a plurality of array segments, and wherein the plurality of array segments is coupled to the first articulating member; and
moving, by the first articulating member, at least one of the plurality of array segments such that at least a portion of the plurality of UV light modules are rotated into a second alignment relative to one another.

17. The method of claim 16, further comprising:
moving, by the robotic device, the end effector with the plurality of UV light modules in the second alignment over a surface to be sanitized.

18. The method of claim 16, further comprising:
determining, by a sensor of the robotic device, contours of a surface to be sanitized; and
determining, based on the contours of the surface, a sanitizing alignment of the plurality of UV light modules, wherein the sanitizing alignment is the second alignment.

19. A robotic system, comprising:
a sensor;
an end effector configured to sanitize surfaces, wherein the end effector comprises:
a plurality of array segments, wherein each array segment of the plurality of array segments is coupled to at least one other array segment of the plurality of array segments;
a plurality of UV light modules, wherein each UV light module of the plurality of UV light modules is coupled to a different array segment of the plurality of array segments; and
a first articulating member configured to cause at least one array segment of the plurality of array segments to move relative to at least one other array segment of the plurality of array segments; and
circuitry configured to perform operations comprising:
determining, based on sensor data from the sensor, contours of a surface to be sanitized; and
operating the first articulating member to move at least one of the plurality of array segments such that the plurality of UV light modules are aligned relative to one another based on the determined contours of the surface to be sanitized.

20. The robotic system of claim 19, wherein the circuitry is further configured to perform operations comprising:
moving the end effector across the surface after the plurality of UV light modules are aligned relative to one another based on the determined contours of the surface to be sanitized.
